(19) United States
(12) Patent Application Publication
(10) Pub. No.: US 2023/0054778 A1
(43) Pub. Date: Feb. 23, 2023

(54) HOLLOW DRILL FOR MEDICAL USE

(71) Applicant: BIC TOOL CO., LTD., Saihaku-gun, Tottori (JP)

(72) Inventors: Koichi Arai, Saihaku-gnn, Tottori (JP); Giichi Arai, Saihaku-gun, Tottori (JP); Katsuyo Kimura, Saihaku-gun, Tottori (JP); Satoshi Teshima, Saihaku-gun, Tottori (JP)

(73) Assignee: BIC TOOL CO., LTD., Saihaku-gun, Tottori (JP)

(21) Appl. No.: 17/619,229

(22) PCT Filed: Dec. 26, 2020
(86) PCT No.: PCT/JP2020/049048

§ 371 Date: Dec. 14, 2021

(30) Foreign Application Priority Data
May 14, 2020 (JP) 2020-085351

(51) Int. Cl. A61B 17/16

(52) U.S. Cl. CPC A61B 17/1615

(57) ABSTRACT

To design a novel tip end shape that performs most of the drilling process so as to prevent chips from entering the gap between the hollow drill bit and the small-diameter drill bit fitted into the hollow portion of the hollow drill bit as well as to provide a structure of the outer periphery so as to allow chips to be discharged toward the outer periphery and prevent them from entering the gap portion.

A hollow drill bit for medical use of the present invention is characterized in comprising a hollow portion that penetrates the axial center in the axial direction from the tip end to the distal end; and four cutting edges at the tip end section, wherein arc-shaped outer peripheral grooves are helically formed in the cylinder-shaped outer peripheral portion.

### FIELD OF THE INVENTION

The present invention relates to a hollow drill bit for medical use.

### BACKGROUND OF THE INVENTION

A hollow drill bit for medical use (hereinafter, simply referred to as “hollow drill bit”) is a drill bit used mainly by orthopedic surgeons for reconstruction of bones, joints, ligaments or the like within the human body, and is directed to drilling holes in bones (See FIGS. 11 to 14a and 14b ). This drill bit is used in such a method that first, the elongated rod-like long drill bit (hereinafter, simply referred to as “small-diameter drill bit”) with the drill-like cutting edges arranged at the tip end, which is called guide pin or guide wire and which is slightly thinner than the hollow portion, drills a hole in the subject bone or joint or the like, and then is fixed thereon, and is inserted in the center (hollow portion) of the hollow drill bit, and using this as a guide, drills the hole by rotation such that the hole is expanded to the intended diameter (external diameter of the hollow drill bit).

In a hollow drill bit for medical use, the present applicant has proposed a drill bit for medical use with a characterized tip end shape in order to enhance the cutting ability of the tip end of the hollow drill bit; the problem to be solved by which being to provide a drill bit for medical use that is resilient to abrasion and excellent in cutting ability by defining the shape for each cutting edge, and based on the shape, creating and arranging two or more cutting edges on the circumference of the drill bit, from the inner end of the tip end to the outer periphery (See Japanese Unexamined Patent Application Publication No. 2018-108223). However, the medical industry has pointed out that when the hollow drill bit (D1) is fixed with the small-diameter drill bit (D2) at the center of rotation of the hollow drill bit (D1) and then the hollow drill (D1) is rotated to drill a hole in the bone (T), bone tissue, which corresponds to cutting chips, may not be completely discharged to the outside of the hollow drill bit (D1) and may enter the subtle gap between the small-diameter drill bit (D2) and the hollow drill bit (D1) (See FIGS. 11 to 14a and 14b).

If the bone tissue enters this gap (GA), following issues may occur:
the hollow drill bit (D1) is impeded from rotating.
the fixed small-diameter drill bit (D2) starts co-rotating, and thus starts drilling unexpectedly.
if the small-diameter drill bit (D2) inadvertently continues drilling the bone (T), the small-diameter drill bit (D2) may drill through the bone (T) and cause unexpected accidents such as damage to surrounding nerve.

The operating physician must pay careful attention upon drilling the bone such that the small-diameter drill bit (D2) fitted into the hollow portion (H) (See FIG. 13) of the hollow drill bit (D1) will not rotate. There are said to be some cases where the physician performs an operation while holding the small-diameter drill bit (D2) with his/her hand to prevent it from rotating.

The present inventors have designed a cutting edge shape that allows the bone tissue to be discharged to the outside of the hollow drill bit as chips while ensuring a gap like a conventional drill bit since when the small-diameter drill bit is fixed and the hollow drill bit is rotated, the hollow drill bit does not rotate smoothly unless there is an appropriate gap therebetween, and the present inventors have earnestly considered to solve the problems.

The present inventors have studied the conventional drill bit shape, and as a result, they have found that due to the structure in which the inner peripheral hollow portion of the cutting edges at the tip end is used at the beginning of drilling process, if the cutting edges at the tip end have poor cutting ability, chips may not be discharged to the side of the outer periphery and may enter the gap between the hollow portion and the small-diameter drill bit (FIGS. 12 to 13).

With respect to the invention described in Japanese Unexamined Patent Application Publication No. 2018-108223 (FIGS. 14a and 14b), where FIG. 14a is a view when viewed from a slightly left side of the front of the tip end, and FIG. 14b is a top view thereof. In FIG. 14a, each cutting edge (15P) has a relief angle (β₂) on the back surface. The relief angle (β₂) is set to satisfy 1°<β₂<20°. The tip end (13P) is provided with chip discharging grooves (21P) from the inner periphery end (18P) to the outer periphery (19P). The chip discharging grooves (21P) have helixes in the axial direction. The angle of the helix (helix angle (β)) of each of the chip discharging grooves (21P) is set to satisfy 5°<δ<45°. The chip discharging grooves (21P) are connected to the cylinder-shaped portion (16P) so that chips are discharged to the outer periphery (19P) at the time of drilling. Each cutting edge (15P) has a relief angle (β₂) on the back surface and chip discharging grooves (21P) have helixes so that a rake angle (θ₃) is formed on the forward surface of each cutting edge. The rake angle (θ₃) on the forward surface of each cutting edge is set to satisfy 2°<θ₃<20°. Gash pockets (22P) are provided at the side of the inner periphery end (18P) of the cutting edges (15P). The gash pockets (22P) are provided to provide clear rakes and facilitate the discharge of chips. In short, the invention of Japanese Unexamined Patent Application Publication No. 2018-108223 has a structure in which the inner peripheral hollow portion of the cutting edges at the tip end is used at the beginning of drilling process, and thus cannot solve these problems mentioned earlier.

### SUMMARY OF THE INVENTION

### Problems to Be Solved by the Invention

For this reason, the object of the present invention is to design a novel tip end shape that performs most of the drilling process so as to prevent chips from entering the gap between the hollow drill bit and the small-diameter drill bit fitted into the hollow portion of the hollow drill bit as well as to provide a structure of the outer periphery so as to allow chips to be discharged toward the outer periphery and prevent them from entering the gap portion.

### Means for Solving the Problem

The invention according to a first aspect relates to a hollow drill bit for medical use comprising a hollow portion that penetrates the axial center in the axial direction from the tip end to the distal end; and four cutting edges at the tip end section, wherein arc-shaped outer peripheral grooves are helically formed in the cylinder-shaped outer periphery.

The invention according to a second aspect relates to a hollow drill bit for medical use according to the first aspect, wherein the chip discharging grooves for discharging chips generated by the cutting edges during the use of the hollow drill bit extend in a twisting manner in the direction from the tip end to the distal end of the hollow drill bit, wherein the chip discharging grooves traverse the four cutting edges and the outer peripheral grooves, and wherein the helical direction of the outer peripheral grooves is same as the twisting direction of the chip discharging grooves.

The invention according to a third aspect relates to the hollow drill bit for medical use according to the first or second aspect, wherein the four cutting edges have a linear shape inclined from the outer periphery to the center of the hollow drill bit, wherein two cutting edges among the four cutting edges that are formed at a distance from the center are main cutting edges, wherein the other two cutting edges that are formed 90° or more rearward from the main cutting edges on a straight line through the center are sub cutting edges, wherein the sub cutting edges are provided with gash pockets, and wherein the helical direction of the outer peripheral grooves is a forward direction.

The invention according to a fourth aspect relates to the hollow drill bit for medical use according to the first, second or third aspect, wherein the two main cutting edges consist of a main cutting edge 1 and a main cutting edge 2, wherein the main cutting edge 1 and the main cutting edge 2 are formed point-symmetrically in the same manner, wherein the main cutting edge 1 is formed starting from the cylinder-shaped portion of the outer periphery, and wherein the main cutting edge 2 is formed starting from the outer peripheral grooves and is formed to be shorter than the main cutting edge 1.

The invention according to a fifth aspect relates to the hollow drill bit for medical use according to any one of the first to fourth aspects, wherein the two sub cutting edges consist of a sub cutting edge 1 and a sub cutting edge 2, wherein the sub cutting edge 1 and the sub cutting edge 2 are positioned 90° or more rearward of the main cutting edge 1 and the main cutting edge 2 relative to a rotational direction of the hollow drill bit, and wherein the sub cutting edge 1 and the sub cutting edge 2 are formed on a same straight line in a radial direction in the cylinder-shaped portion.

The invention according to a sixth aspect relates to the hollow drill bit for medical use according to any one of the first to fifth aspects, wherein an angle α1 of the main cutting edges inclined toward the center and an angle α2 of the sub cutting edges inclined toward the center satisfy a relationship of 0°<α1<α2.

The invention according to a seventh aspect relates to the hollow drill bit for medical use according to any one of the first to sixth aspects, wherein an inclined angle (Θ) made by the main cutting edge 1 and the main cutting edge 2 is formed in a direction in which chips are discharged to the side of the outer periphery due to rotation of the hollow drill bit, and as a result, chips generated by the drilling process of the main cutting edge 1 are discharged from the side of the rake of the main cutting edge 1 to the chip discharging grooves 1 via outer peripheral grooves, wherein the chip discharging grooves comprise chip discharging grooves 1 provided at the side of the rakes of the sub cutting edges and chip discharging grooves 2 provided on side surfaces at the side of heels, wherein the chip discharging grooves 1 are gently connected in the direction from the side of the heels of the main cutting edges at the tip end of the hollow drill bit to the distal end of the hollow drill bit, and are connected to the helical outer peripheral grooves continuing from the side of the rakes of the main cutting edges with a twist toward the side of a shank, which is at the distal end of the hollow drill bit, and wherein the chip discharging grooves 2 have a same helix direction and a same helix angle as the chip discharging grooves 1.

The invention according to an eighth aspect relates to a composite drill bit comprising a small-diameter drill bit fitted into the hollow portion of the hollow drill bit according to any one of the first to seventh aspects.

The invention according to a ninth aspect relates to the hollow drill bit for medical use according to the first aspect, wherein grooves forming outer peripheral cutting edges with a depth equivalent to or shallower than the depth of the outer peripheral grooves extend in a twisting manner in the direction from the tip end to the distal end of the hollow drill bit, wherein the grooves forming outer peripheral cutting edges transverse the four cutting edges and the outer peripheral grooves, and wherein the helical direction of the outer peripheral grooves is same as the twisting direction of the grooves forming outer peripheral cutting edges.

The invention according to a tenth aspect relates to the hollow drill bit for medical use according to the ninth aspect, wherein two cutting edges among the four cutting edges that are formed at a distance from the center are main cutting edges, wherein the other two cutting edges that are formed 90° or more rearward from the main cutting edges on a straight line through the center are sub cutting edges, wherein an angle α1 of the main cutting edges inclined toward the center and an angle α2 of the sub cutting edges inclined toward the center satisfy a relationship of α1=α2=0, wherein the sub cutting edges are provided with gash pockets, and wherein the helical direction of the outer peripheral grooves is the positive direction.

The invention according to a eleventh aspect relates to the hollow drill bit for medical use according to the ninth or tenth aspect, wherein the two main cutting edges consist of a main cutting edge 1 and a main cutting edge 2, wherein the main cutting edge 1 and the main cutting edge 2 are formed point-symmetrically in the similar manner, wherein the main cutting edge 1 is formed starting from the cylinder-shaped portion of the outer periphery, and wherein the main cutting edge 2 is formed starting from the outer peripheral grooves and is formed to be shorter than the main cutting edge 1.

The invention according to a twelfth aspect relates to the hollow drill bit for medical use according to any one of the ninth to twelfth aspects, wherein the two sub cutting edges consist of a sub cutting edge 1 and a sub cutting edge 2, wherein the sub cutting edge 1 and the sub cutting edge 2 are positioned 90° or more rearward of the main cutting edge 1 and the main cutting edge 2 relative to a rotational direction of the hollow drill bit, and wherein the sub cutting edge 1 and the sub cutting edge 2 are formed on a same straight line in a radial direction in the cylinder-shaped portion.

The invention according to a thirteenth aspect relates to a composite drill bit comprising a small-diameter drill bit fitted into the hollow portion of the hollow drill bit according to any one of the eighth to twelfth aspects.

### Effects of the Invention

The invention according to the first aspect has constructional characteristics that a hollow drill bit for medical use comprises a hollow portion that penetrates the axial center in the axial direction in the direction from the tip end to the distal end; and four cutting edges at the tip end section, that arc-shaped outer peripheral grooves are helically formed in a cylindrical-shaped outer periphery, which produces an advantageous effect of allowing chips to be discharged toward the outer periphery and preventing them from entering the gap portion.

The invention according to the second aspect has characteristics that chip discharging grooves for discharging chips generated by the cutting edges during the use of the hollow drill bit extend in a twisting manner in the direction from the forward end to the distal end of the hollow drill bit, that the chip discharging grooves traverse the four cutting edges and the outer peripheral grooves, and that the helical direction of the chip discharging grooves is same as the twisting direction of the chip discharging grooves, which produces an advantageous effect of allowing chips to be discharged toward the outer periphery and preventing them from entering the gap portion.

The invention according to the third aspect has constructional characteristics that the four cutting edges have a linear shape inclined from the outer periphery to the center of the hollow drill bit, where two cutting edges among the four cutting edges that are formed at a distance from the center are main cutting edges, that the other two cutting edges that are formed 90° or more rearward from the main cutting edges on a straight line through the center are sub cutting edges, that the sub cutting edges are provided with gash pockets, and that the helical direction of the outer peripheral grooves is a forward direction, which provides a structure in which chips generated at the time of drilling will not move in the direction toward the inner periphery of the hollow drill bit, but will move in the direction toward the outer periphery under the additional influence of centrifugal force when the hollow drill bit is rotated, and generates an advantageous effect of making it difficult for chips to move to the hollow portion of the hollow drill bit. Further, the sub cutting edges are provided with gash pockets, which produces an advantageous effect of keeping chips in the gash pockets in case where chips accumulate, and preventing them from entering the gap.

The invention of the fourth aspect has constructional characteristics that the two main cutting edges consist of a main cutting edge 1 and a main cutting edge 2, that the main cutting edge 1 and the main cutting edge 2 are formed point-symmetrically in the similar manner, that the main cutting edge 1 is formed starting from the cylinder-shaped portion of the outer periphery, and that the main cutting edge 2 is formed starting from the outer peripheral grooves and is formed to be shorter than the main cutting edge 1. The invention according to the fifth aspect has constructional characteristics that the two sub cutting edges consist of the sub cutting edge 1 (14) and the sub cutting edge 2 (15), that the sub cutting edge 1 (14) and the sub cutting edge 2 (15) are positioned 90° or more rearward of the main cutting edge 1 (12) and the main cutting edge 2 (13) relative to the rotational direction of the hollow drill bit (11), and that the sub cutting edge 1 (14) and the sub cutting edge 2 (15) are formed on a same straight line in a radial direction in the cylinder-shaped portion. Therefore, the drilling process by the inner periphery are carried out by the main cutting edge 1 and the main cutting edge 2, and the sub cutting edges are not involved. This produces an advantageous effect of preventing chips from flowing into the hollow portion by the inclined angle made on the main cutting edge 1 and the main cutting edge 2.

The invention according to the sixth aspect has a configuration that the angle α1 of the main cutting edges inclined toward the center and the angle α2 of the sub cutting edges inclined toward the center satisfy the relationship of 0°<a1<a2 so that the sub cutting edges are not involved in the drilling process except the outer periphery at the side of the tip end, which produces an advantageous effect of preventing chips from entering the gap portion.

The invention according to the seventh aspect has characteristics that the inclined angle (Θ) made by the main cutting edge 1 and the main cutting edge 2 is formed in a direction in which chips are discharged to the side of the outer periphery due to rotation of the hollow drill bit, and as a result, chips generated by the drilling process of the main cutting edge 1 are discharged from the side of the rake of the main cutting edge 1 to the chip discharging grooves 1 via outer peripheral grooves,
that the chip discharging grooves 1 are gently connected in the direction from the side of the heels of the main cutting edges at the tip end of the hollow drill bit to the distal end of the hollow drill bit, and are connected to the helical outer peripheral grooves continuing to the side of the rakes of the main cutting edges with a twist toward the side of the shank, which is at the distal end of the drill bit, and
that the chip discharging grooves 2 have a same helix direction and a same helix angle as the chip discharging grooves 1, which produces an advantageous effect of ensuring chips to be discharged from the chip discharging grooves while reducing the friction with the outer periphery of the hollow drill bit, and allowing a smooth drilling process.

The composite drill bit of the invention according to the eighth aspect is provided with a configuration that the small-diameter drill bit fitted into the hollow drill bit according to any one of the first to sixth aspects, and thus the operating physician does not need to pay careful attention upon drilling the bone such that the small-diameter drill bit fitted into the hollow portion of the hollow drill bit will not rotate, nor does he/she need to perform an operation while holding the small-diameter drill bit with their hand to prevent it from rotating.

The composite drill bit of the invention according to the ninth aspect has characteristics that the grooves forming an outer peripheral cutting edges with a depth equivalent to or shallower than the depth of the outer peripheral grooves extend in a twisting manner in the direction from the tip end to the distal end of the hollow drill bit, that the grooves forming outer peripheral cutting edges transverse the four cutting edges and the outer peripheral grooves, and that the helical direction of the outer peripheral grooves is same as the twisting direction of the grooves forming outer peripheral cutting edges, which produces an advantageous effect of allowing chips to be discharged toward the outer periphery and preventing them from entering the gap portion.

The invention according to the tenth aspect has constructional characteristics that two cutting edges among the four cutting edges that are formed at a distance from the center are main cutting edges, that the other two cutting edges that are formed 90° or more rearward from the main cutting edges on a straight line through the center are sub cutting edges, that an angle α1 of the main cutting edges inclined toward the center and an angle α2 of the sub cutting edges inclined toward the center satisfy a relationship of α1=α2=0, that the sub cutting edges are provided with gash pockets, and that the helical direction of the outer peripheral grooves is the positive direction, which provides a structure in which chips generated at the time of drilling will not move in the direction toward the inner periphery of the hollow drill bit , but will move in the direction toward the outer periphery under the additional influence of centrifugal force when the hollow drill bit is rotated, and generates an advantageous effect of making it difficult for chips to move to the hollow portion of the hollow drill bit. Further, the sub cutting edges are provided with gash pockets, which produces an advantageous effect of keeping chips in the gash pockets in case where chips accumulate, and preventing them from entering the gap.

The invention of the eleventh aspect has constructional characteristics that the two main cutting edges consist of a main cutting edge 1 and a main cutting edge 2, that the main cutting edge 1 and the main cutting edge 2 are formed point-symmetrically in the similar manner, that the main cutting edge 1 is formed starting from the cylinder-shaped portion of the outer periphery, and that the main cutting edge 2 is formed starting from the outer peripheral grooves and is formed to be shorter than the main cutting edge 1. Further, the invention according to the twelfth aspect has constructional characteristics that the two sub cutting edges consist of the sub cutting edge 1 and the sub cutting edge 2, that the sub cutting edge 1 and the sub cutting edge 2 are positioned 90° or more rearward of the main cutting edge 1 and the main cutting edge 2 relative to the rotational direction of the hollow drill bit, and that the sub cutting edge 1 and the sub cutting edge 2 are formed on a same straight line in a radial direction in the cylinder-shaped portion. As mentioned above, a structure is established in which the main cutting edge 1 is used to penetrate into the bone (to ensure a hole with a diameter as large as the drill diameter), and then the sub cutting edges 1 and 2 are used to perform most of the drilling process, and the main cutting edge 4 is used to complement the main cutting edge 1 as it penetrates into the bone. This produces an advantageous effect of preventing chips from flowing into the hollow portion by the inclined angle made on the main cutting edge 1 and the main cutting edge 2.

The invention of the thirteenth aspect is provided with a configuration that the small-diameter drill bit fitted into the hollow portion of the hollow drill bit according to any one of the eighth to twelfth aspects, and thus the operating physician does not need to pay careful attention upon drilling the bone such that the small-diameter drill bit fitted into the hollow portion of the hollow drill bit will not rotate, nor does he/she need to perform an operation while holding the small-diameter drill bit with their hand to prevent it from rotating.

### BRIEF DESCRIPTION OF DRAWINGS

FIG. 1 is a front view of the tip end of the hollow drill bit for medical use according to Embodiment 1 of the present invention.

FIG. 2 is a bottom view of the drill bit according to Embodiment 1 of FIG. 1.

FIGS. 2-1 is a bottom view of the drill bit according to Embodiment 1 of FIG. 1, and is an explanatory view of the tip end of the drill bit.

FIG. 3 is a right side view of the drill bit according to Embodiment 1 of FIG. 1.

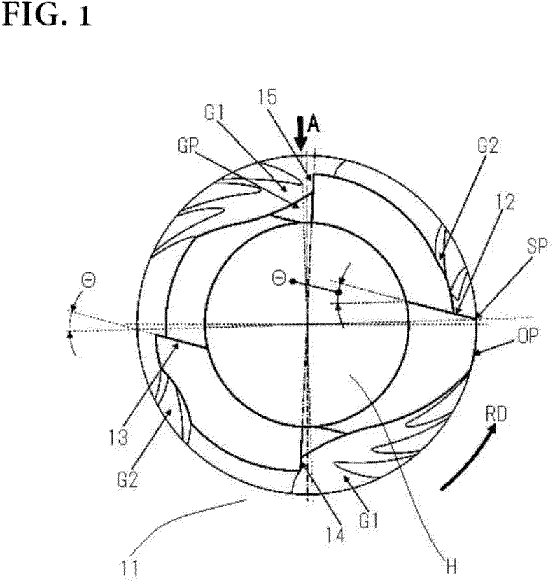

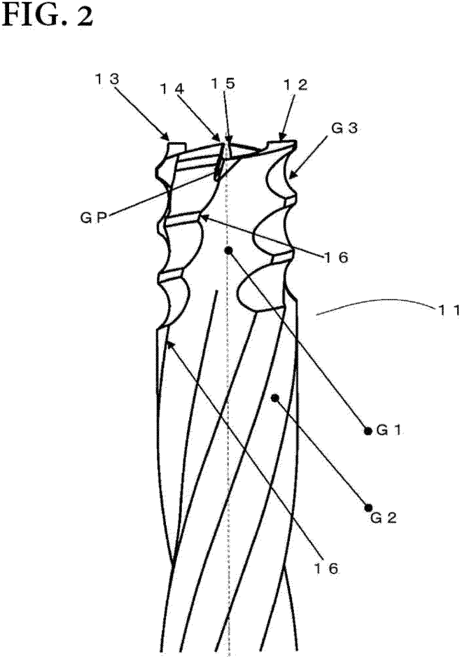

FIGS. 2-1 is a right side view of the drill bit according to Embodiment 1 of FIG. 1, and is an explanatory view of the tip end of the drill bit.

FIG. 4 is a top view of the drill bit of Embodiment 1 of FIG. 1 when viewed from a direction perpendicular to and oblique to the main cutting edges.

FIG. 5 is a perspective view of the drill bit according to Embodiment 1 of FIG. 1.

FIG. 6 is a perspective view of a different section of Embodiment 1 from the perspective view of FIG. 5.

FIG. 7 is a perspective view of the entirety of the drill bit of Embodiment 1 of FIG. 1, from the tip end to the distal end.

FIGS. 7-1 is an explanatory view of an additional embodiment in which a small-diameter drill bit is fitted into the hollow portion of the drill bit of FIG. 1.

FIG. 8 is a side view of an example of a hollow drill bit for medical use according to another Embodiment 2 of the present invention.

FIG. 9 is a perspective view of the drill bit according to Embodiment 2 of FIG. 8.

FIG. 10 is a perspective view of a different section of the drill bit from the perspective view of FIG. 9.

FIG. 11 is an explanatory view illustrating the scene in which a hole is being made in a bone using a composite drill bit of a combination of a hollow drill bit of a conventional art and a small-diameter drill bit fitted in the hollow portion thereof.

FIG. 12 is a partial enlarged view illustrating the tip end of the conventional composite drill bit of FIG. 11.

FIG. 13 is an explanatory view illustrating the tip end of the conventional hollow drill bit.

FIG. 14a is a view illustrating a hollow drill bit according to Japanese Unexamined Patent Application Publication No. 2018-108223, where FIG. 14a is a view viewed from a slightly left side of the front of the tip end.

FIG. 14b is a view illustrating a hollow drill bit according to Japanese Unexamined Patent Application Publication No. 2018-108223, where FIG. 14b is a top view thereof.

FIG. 15 is a front view of the tip end of the hollow drill bit for medical use according to Embodiment 4 of the present invention.

FIG. 16 is a bottom view of the drill bit of Embodiment 4 of FIG. 15.

FIG. 17 is a perspective view of the tip end of the hollow drill bit for medical use according to Embodiment 4 when viewed from direction A of FIG. 15.

FIG. 18 is a perspective view of the tip end of the hollow drill bit for medical use bit according to Embodiment 4 when viewed from direction B of FIG. 15.

FIG. 19 is a reference view of the hollow drill bit for medical use according to Embodiment 4 when viewed from direction B of FIG. 15, and shows a structure on the assumption that a deep hole will be made.

FIG. 20 is a bottom view of the tip end of the hollow drill bit for medical use according to Embodiment 4 when viewed from direction B of FIG. 15.

### DETAILED DESCRIPTION OF THE INVENTION

Embodiments of a hollow drill bit for medical use are described in detailed below with reference to the accompanying drawings.

### Embodiment 1

FIG. 1 is a front view of the tip end of the hollow drill bit for medical use according to Embodiment 1 of the present invention. FIG. 2 is a bottom view of the drill bit of FIG. 1.

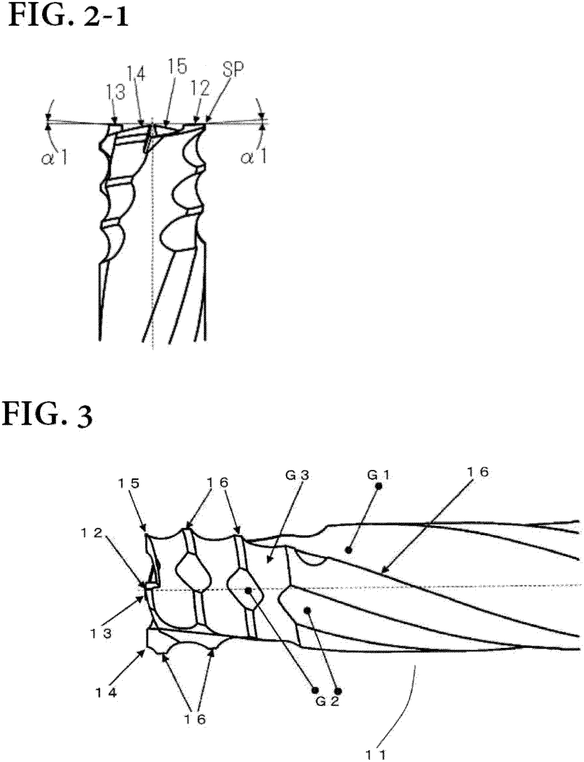

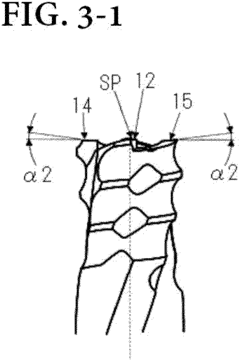

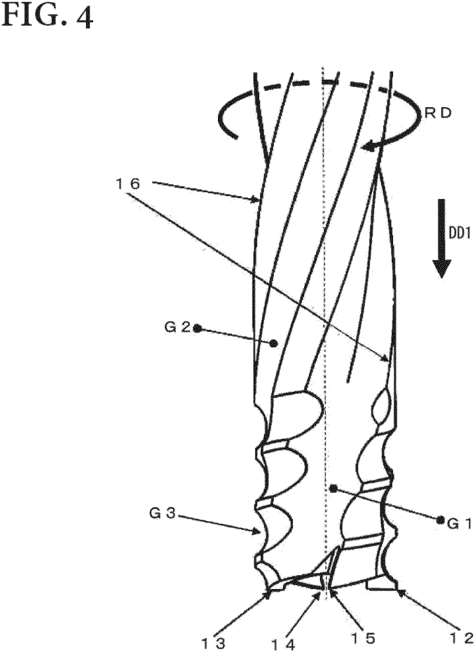

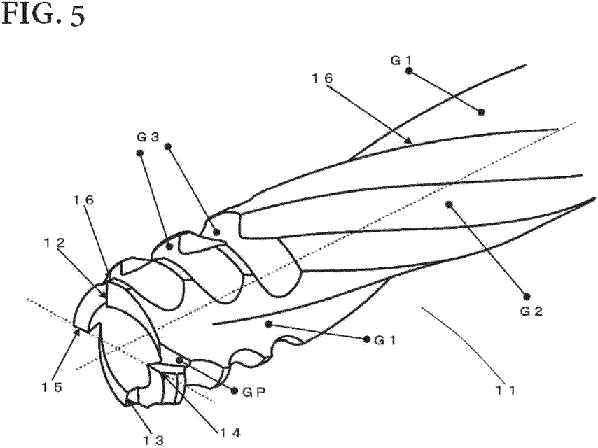

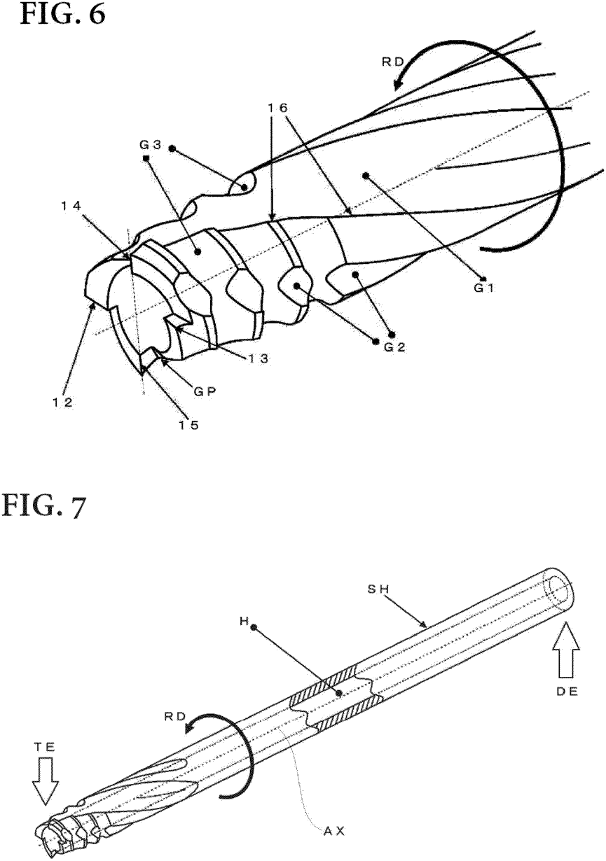

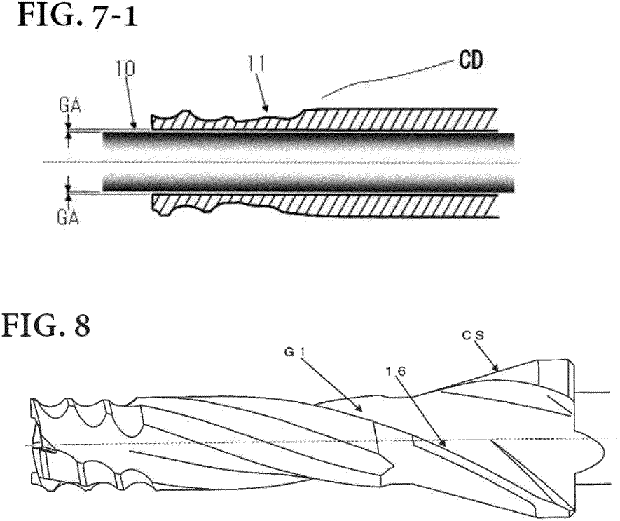

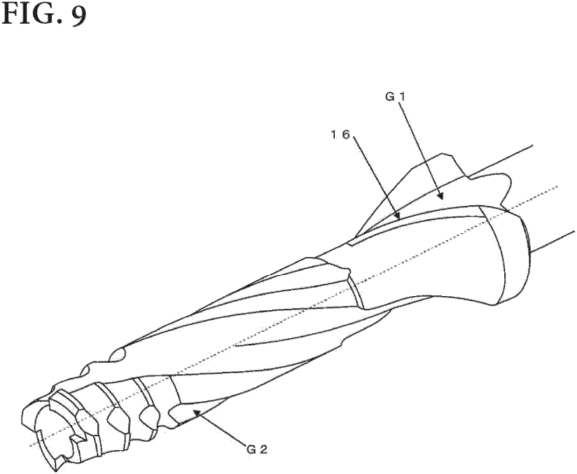

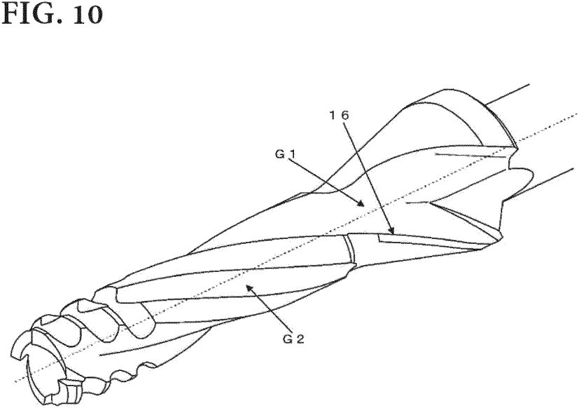

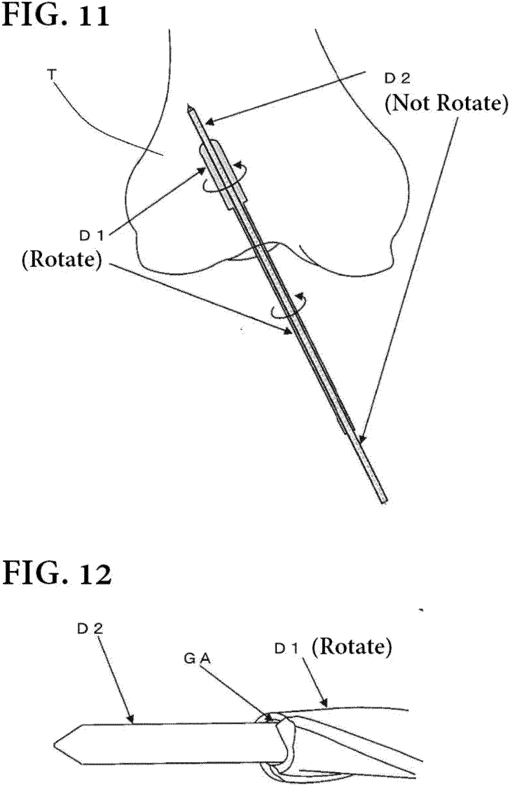

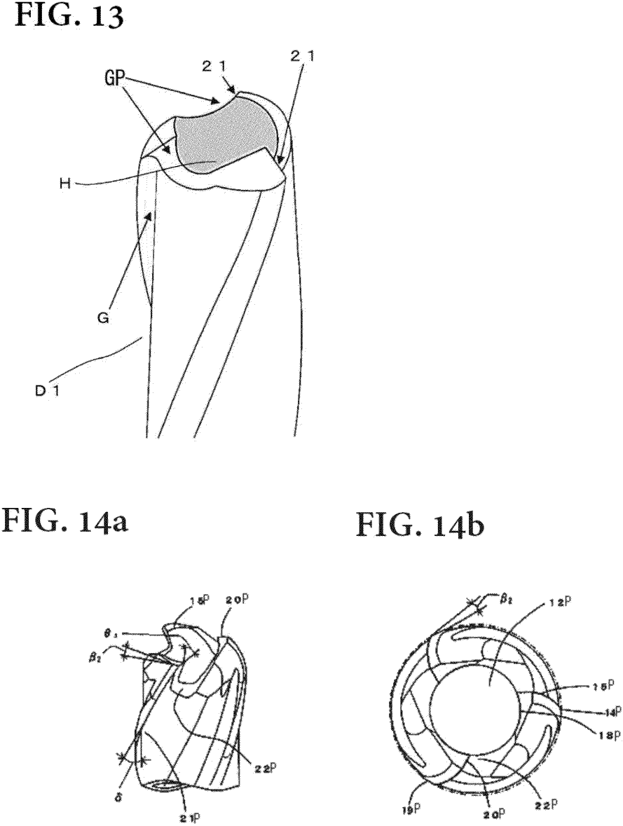

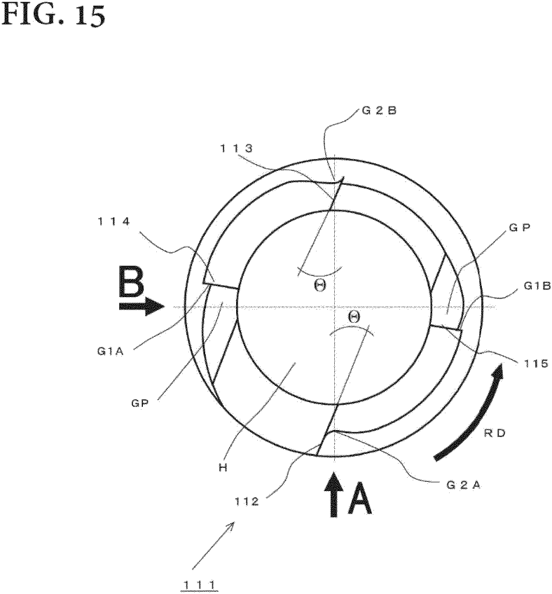

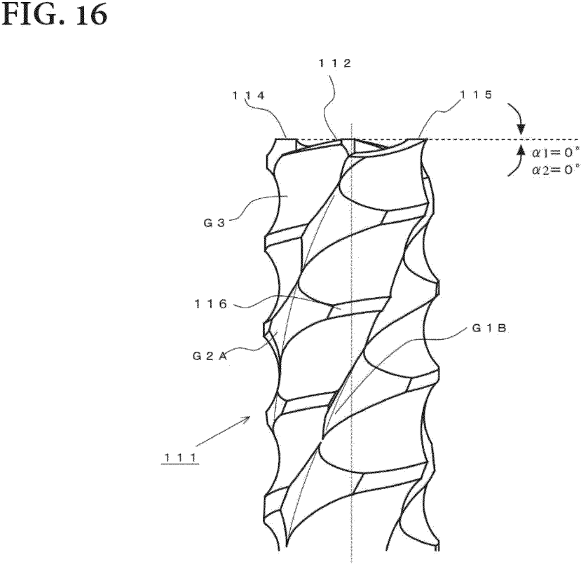

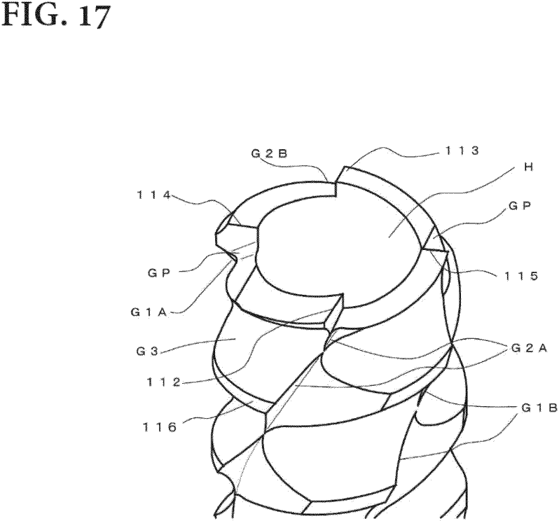

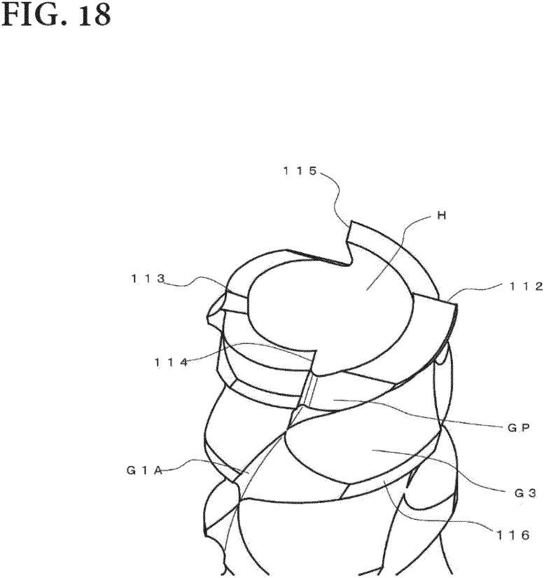

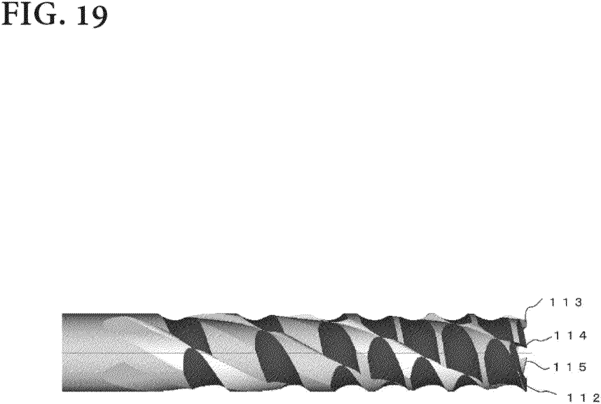

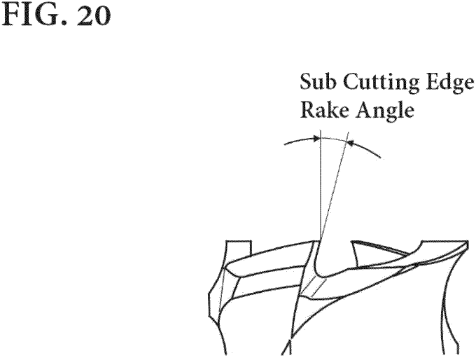

FIGS. 2-1 is a bottom view of the drill bit of FIG. 1, and is an explanatory view of the tip end of the drill bit. FIG. 3 is a right side view of the drill bit of FIG. 1. FIGS. 3-1 is a bottom view of the drill bit of FIG. 1, and is an explanatory view of the tip end of the drill bit. FIG. 4 is a top view of the drill bit of FIG. 1 when viewed from a direction perpendicular to and oblique to the main cutting edges. FIG. 5 is a perspective view of the drill bit in FIG. 1. FIG. 6 is a perspective view illustrating a different section from the perspective view of FIG. 5. FIG. 7 is a perspective view of the entirety of the drill bit of FIG. 1, from the tip to the distal tip.

Referring to FIG. 7, the hollow drill bit (11) of this embodiment is provided with a hollow portion (H) that penetrates the axial center (AX) from the tip end (TE) to the distal end (DE) in the axial direction.

Referring to FIGS. 1 and 7, the hollow drill bit (11) of this embodiment has four cutting edges (i.e. a main cutting edge 1 (12), a main cutting edge 2 (13), a sub cutting edge 1 (14), and a sub cutting edge 2 (15)) at the tip end (TE) section, and arc-shaped outer peripheral grooves (G3) are helically formed in a cylinder-shaped outer periphery (OP).

Further, chip discharging grooves (i.e. chip discharging grooves 1 (G1) and chip discharging grooves 2 (G2)) for discharging chips generated by the cutting edges mentioned above (i.e. the main cutting edge 1 (12), the main cutting edge 2 (13), the sub cutting edge 1 (14), and the sub cutting edge 2 (15)) during the use (during the bone-drilling operation) of the hollow drill bit (11) of this embodiment extend in a twisting manner in the direction from the tip end (TE) of the hollow drill bit (11) to the distal end (DE) (that is, toward the distal end (DE)).

Referring to FIGS. 2 and 3, the chip discharging grooves (i.e. the chip discharging grooves 1 (G1) and the chip discharging grooves 2 (G2)) traverse the four cutting edges (i.e. the main cutting edge 1 (12), the main cutting edge 2 (13), the sub cutting edge 1 (14), and the sub cutting edge 2 (15)) and the outer peripheral grooves (G3).

In the hollow drill bit (11) of this embodiment, the helical direction of the outer peripheral grooves (G3) is same as the twisting direction of the chip discharging grooves (i.e. the chip discharging grooves 1 (G1) and the chip discharging grooves 2 (G2)), and the helical direction is the positive direction (in other words, the outer peripheral grooves (G3) are twisted to form a Z-shape when the tip end (TE) of the hollow drill bit (11) of this embodiment is viewed from above).

Referring to FIG. 1, the four cutting edges (i.e. the main cutting edge 1 (12), the main cutting edge 2 (13), the sub cutting edge 1 (14), and the sub cutting edge 2 (15)) have a linear shape inclined from the outer periphery (OP) toward the center of the hollow drill bit (11), and two cutting edges among the four cutting edges (i.e. the main cutting edge 1 (12), the main cutting edge 2 (13), the sub cutting edge 1 (14), and the sub cutting edge 2 (15)) that are formed at a distance from the center are main cutting edges (the main cutting edge 1 (12) and the main cutting edge 2 (13)), and the other two cutting edges that are formed 90° or more rearward of the main cutting edges (the main cutting edge 1 (12) and the main cutting edge 2 (13)) on a straight line through the center are sub cutting edges (the sub cutting edge 1 (14), and the sub cutting edge 2 (15)).

Further, referring to FIG. 1 and FIG. 2, the sub cutting edges (the sub cutting edge 1 (14), and the sub cutting edge 2 (15)) are provided with recesses referred to as gash pockets (GP) at the side of the rakes. These gash pockets (GP) are not provided for the drilling purpose. These gash pockets (GP) have an effect of preventing chips from entering the gap (GA) (See conventional FIG. 12 and FIG. 13, other than FIGS. 7-1) between the hollow drill bit (11) and the small-diameter drill bit (D2) (FIG. 11 and FIG. 12) fitted in the hollow portion of the hollow drill bit (11) by keeping chips in the gash pockets (GP) in case where chips accumulate due to decreased cutting ability of the hollow drill bit (11) during the drilling operation. Such configuration has an advantageous effect of preventing the heat generation during the drilling process and achieving an improved cutting ability. In this embodiment, these gash pockets (GP) are provided starting from the inner periphery of the hollow drill bit (11) to the chip discharging grooves 1 (G1). These gash pockets (GP) do not have areas enlarging toward the inner periphery, unlike those in FIG. 13 and FIGS. 14a and 14b, but they are configured to have areas gradually enlarging from the ridge of the inner periphery toward the outer periphery. Therefore, these gash pockets (GP) can prevent chips from occupying the gap (GA).

Also, in this embodiment, the two main cutting edges mentioned above comprise the main cutting edge 1 (12) and the main cutting edge 2 (13), where the main cutting edge 1 (12) and the main cutting edge 2 (13) are formed point-symmetrically in the similar manner.

The main cutting edge 1 (12) is formed starting from the cylinder-shaped portion of the outer periphery (OP). In contrast, the main cutting edge 2 (13) is formed starting from the outer peripheral grooves (G3) and is formed to be shorter than the main cutting edge 1 (12). As exemplified in FIG. 1, the main cutting edge 1 (12) and the main cutting edge 2 (13) have linear portions with an inclined angle (Θ) inclined away from the center. Having this inclined angle (Θ), the hollow drill bit (11) has a configuration that chips generated at the time of drilling the bone will not move in the direction toward the inner periphery of the hollow drill bit (11), but will move in the direction toward the outer periphery under the additional influence of centrifugal force when the hollow drill bit (11) in the present embodiments is rotated, and generates an advantageous effect of making it difficult for chips to move to the hollow portion (H) of the hollow drill bit (11). Further, the main cutting edge 1 (12) and the main cutting edge 2 (13) of the hollow drill bit (11) are configured such that the chip discharging grooves 1 (G1) and the chip discharging grooves 2 (G2) are not provided on the side of the rakes of the main cutting edge 1 (12) and the main cutting edge 2 (13).

The two sub cutting edges mentioned above comprise the sub cutting edge 1 (14) and the sub cutting edge 2 (15), where the sub cutting edge 1 (14) and the sub cutting edge 2 (15) are positioned 90° or more rearward of the main cutting edge 1 (12) and the main cutting edge 2 (13) relative to the rotational direction of the hollow drill bit (11), and where the sub cutting edge 1 (14) and the sub cutting edge 2 (15) are formed on a same straight line in a radial direction in the cylinder-shaped portion.

The sub cutting edge 1 (14) and the sub cutting edge 2 (15) of the hollow drill bit (11) in the embodiments do not serve in the drilling process, except the outer periphery on the side of the tip end. However, the sub cutting edge 1 (14) and the sub cutting edge 2 (15) are necessary elements to achieve a stable drilling process in a method of drilling helically, starting with the main cutting edge 1 (12).

Referring to FIGS. 1, 2, 2-1, 3 and 3-1, the angle α1 inclined toward the center of the main cutting edges (the main cutting edge 1 (12) and the main cutting edge 2 (13)) and the angle α2 inclined toward the center of the sub cutting edges satisfy the relationship of 0°<α1<α2. The angle α1 is set to be within the range of 0°<α1 <5°, and the angle α2 is set to be equal to or greater than the angle α1 and less than 10°. Preferably, the angle α1 and the angle α2 are set to satisfy the relationships of 1°<α1<3° and 5°<α2<7°.

Next, the chip discharging grooves (i.e. the chip discharging grooves 1 (G1) and the chip discharging grooves 2 (G2)) of the hollow drill bit (11) in the embodiments are described in details. The chip discharging grooves comprise the chip discharging grooves 1 (G1) provided on the side of the rakes of the sub cutting edges (the sub cutting edge 1 (14) and the sub cutting edge 2 (15)), and the chip discharging grooves 2 (G2) provided on the side surfaces on the side of the heels, as shown in FIG. 1. The chip discharging grooves 1 (G1) are gently connected in the direction from the side of the heels of the main cutting edges (the main cutting edge 1 (12) and the main cutting edge 2 (13)) at the tip end (TE) of the hollow drill bit (11) to the distal end (DE) of the hollow drill bit (11), and are connected to the helical outer peripheral grooves (G3) continuing from the side of the rakes of the main cutting edges (the main cutting edge 1 (12) and the main cutting edge 2 (13)) with a twist toward the side of the shank, which is at the distal end (DE) of the drill bit. The chip discharging grooves 2 (G2) have the same helix direction and the same helix angle as the chip discharging grooves 1 (G1).

In this embodiment, the main cutting edges (the main cutting edge 1 (12) and the main cutting edge 2 (13)) are connected to the helical outer peripheral grooves (G3) continuing to the side of the rakes of the main cutting edges, thereby providing an advantageous effect of allowing chips to be smoothly discharged. The outer peripheral grooves (G3) are formed helically and connected to the side of the rake of the main cutting edge 1 (12), which allows chips generated during the drilling process performed mainly by the main cutting edge 1 (12) to be sent, through the helical outer peripheral grooves (G3) continuing to the side of the rake of the main cutting edge 1 (12), to the chip discharging grooves (G1) and be discharged therefrom. Therefore, chips are discharged mainly from the chip discharging grooves 1 (G1) connected to the side of the rake of the sub cutting edge 1 (14), through the outer peripheral grooves (G3) connected from the side of the rake of the main cutting edge 1 (12). The chip discharging grooves are not provided on the side of the rakes of the main cutting edges (the main cutting edge 1 (12) and the main cutting edge 2 (13)). The helical outer peripheral grooves (G3) on the side of the outer periphery of the cutting edges serve as chip discharging grooves.

Further, the chip discharging grooves 2 (G2) shallower and smaller than the chip discharging grooves 1 (G1) are provided starting from the side surfaces of the side of the heels of the sub cutting edges (the sub cutting edge 1 (14) and the sub cutting edge 2 (15)). As mentioned above, the chip discharging grooves 2 (G2) have the same rake direction and the same rake angle as the chip discharging grooves 1 (G1), and are provided as complementary chip discharging grooves. The chip discharging grooves 2 (G2) are provided on the side surfaces on the side of the heels of the sub cutting edges (the sub cutting edge 1 (14) and the sub cutting edge 2 (15)) and forward of the main cutting edges (the main cutting edge 1 (12) and the main cutting edge 2 (13)), and are arranged at a location synchronized with the location where chips are discharged during the drilling process by the inner periphery of the main cutting edges (the main cutting edge 1 (12) and the main cutting edge 2 (13)).

The helical outer peripheral grooves (G3), the chip discharging grooves 1 (G1), and the chip discharging grooves 2 (G2), which are characteristics of the outer periphery of the hollow drill bit (11) in the embodiments, are provided to reduce the contact area between the outer periphery and the bone, for the hollow drill bit (11) in the embodiments has a configuration with four cutting edges (i.e. the main cutting edge 1 (12), the main cutting edge 2 (13), the sub cutting edge 1 (14), and the sub cutting edge 2 (15)). Therefore, the hollow drill bit (11) in this embodiment produces an excellent effect of reducing the friction due to the contact and suppressing the rise in temperature.

The hollow drill bit (11) of the embodiments is provided with the above-mentioned configuration, and the drilling process starts with the outer periphery of the main cutting edge 1 (12) being brought into contact with the bone, and then the tip of each outer periphery being brought into contact with the bone in the order of the sub cutting edge 1 (14), the main cutting edge 2 (13), and the sub cutting edge 2 (15), and subsequently the main cutting edge 1 (12) and the main cutting edge 2 (13). A hole with a diameter corresponding to the diameter of the hollow drill bit (11) is achieved by the drilling process by the main cutting edge 1 (12). The reference numeral (SP) in FIG. 1 indicates the starting point of the drilling process.

The structure from the tip end to the chip discharging grooves 1 (G1) and the chip discharging grooves 2 (G2) of the hollow drill bit (11) according to this embodiment is illustrated in FIG. 1 to FIG. 6, and has, toward the shank at the side of the distal end (DE), a pipe shape (or tube shape) with the same diameter as the drill diameter of the hollow drill bit (11), as shown in FIG. 7, or a thinner pipe shape (or tube shape) than the drill diameter, and an entire length set depending on the type of the operation.

### Embodiment 2

FIG. 8 is a side view of an example of a hollow drill bit for medical use according to Embodiment 2. FIG. 9 is a perspective view of the drill bit of FIG. 8. FIG. 10 is a perspective view illustrating a different section of the drill bit from the perspective view of FIG. 9.

The hollow drill bit (11) according to Embodiment 2 is provided with counter sinks (CS) near the rearward ends of the chip discharging grooves 1 (G1) to simultaneously perform the burr removal and chamfering process after the drilling process by the drill bit. It should be understood that the hollow drill bit (11) according to this embodiment adopts the same structure as that of the above-mentioned Embodiment 1, except the counter sinks (CS).

### Embodiment 3

Referring to FIG. 7 and FIGS. 7-1, the hollow drill bit (11) of the composite drill bit for medical use (CD) according to Embodiment 3 adopts the same configuration as that of the above-mentioned Embodiment 1 or Embodiment 2, and comprise a small-diameter drill bit (10) fitted into the hollow portion (H) (See FIG. 7) of the hollow drill bit (11) such that the small-diameter drill bit (10) does not rotate together with the hollow drill bit (11). The hollow drill bit (11) may adopt a small-diameter drill bit described in the above-mentioned conventional art or another well-known small-diameter drill bit as the small-diameter drill bit (10).

### Embodiment 4

FIG. 15 is a front view of the tip end of the hollow drill bit for medical use according to an embodiment of the present invention. FIG. 16 is a bottom view of the drill bit of FIG. 15. FIG. 17 is a perspective view of the drill bit of FIG. 15 when the tip end is viewed from a direction substantially parallel to and oblique to the main cutting edges. FIG. 18 is a perspective view of the drill bit of FIG. 15 when the tip end is viewed from a direction substantially parallel to and oblique to the sub cutting edges. FIG. 19 is a left side view of the drill bit of FIG. 15.

It was assumed that the hollow drill bit according to [Embodiment 1] mentioned above has a structure in which a certain thickness can be ensured. Therefore, large chip discharging grooves 1 (G1) were arranged rearward of the main cutting edge 1 (12) and the main cutting edge 2 (13) (and forward of the sub cutting edges) (relative to the rotational direction), and the chip discharging grooves 2 (G2) were complementarily provided in the outer periphery of the drill.

However, with consideration of a case where the same structure is manufactured with a thinner thickness and the drill bit does not have the chip discharging effect or the drilling ability as anticipated, the drill bit has a structure in which chips are not discharged from the chip discharging grooves like in [Embodiment 1], but from the outer peripheral grooves (G3) helically provided in the outer periphery, and in which the angles of the main cutting edge 1 (112) and the main cutting edge 2 (113), and the angles of the sub cutting edge 1 (114) and the sub cutting edge 2 (115) are set to be parallel so that all of the four cutting edges are able to drill, and, based on such structure, the sub cutting edges 1 and 2 have rake angles set and have shapes for performing most of the drilling process.

Conventional chip discharging grooves are provided as grooves forming outer peripheral cutting edges (G1A, G1B, G2A, G2B) to form outer peripheral cutting edges (116) and the depth of the grooves is set to be equivalent to that of the outer peripheral grooves (G3) or shallower than that of the outer peripheral grooves (G3) to allow chips to be led by the outer peripheral grooves (G3) through the helical locus to the rearward end of the drill bit and then discharged therefrom.

Referring to FIG. 15, FIG. 17 and FIG. 18, the hollow drill bit (111) in this embodiment has four cutting edges (i.e. a main cutting edge 1 (112), a main cutting edge 2 (113), a sub cutting edge 1 (114), and a sub cutting edge 2 (115)) at the tip end (TE) section, and arc-shaped outer peripheral grooves (G3) helically formed in a cylindrical-shaped outer periphery (OP).

Further, the grooves forming outer peripheral cutting edges (i.e. the groove forming an outer peripheral cutting edge 1A (G1A), the groove forming an outer peripheral cutting edge 1B (GIB), the groove forming an outer peripheral cutting edge 2A (G2A), and the grooves forming an outer peripheral cutting edge 2B (G2B)) for allowing chips generated by the cutting edges mentioned above (i.e. the main cutting edge 1 (112), the main cutting edge 2 (113), the sub cutting edge 1 (114), and the sub cutting edge 2 (115)) during the use of the hollow drill bit (11) of this embodiment (during the bone-drilling operation) to be led to the outer peripheral grooves (G3) while forming the outer peripheral cutting edges, extend in a twisting manner in the direction from the tip end (TE) of the hollow drill bit (111) to the distal end (DE) (i.e. toward the distal end (DE)).

Referring to FIG. 16, the grooves forming outer peripheral cutting edges (i.e. the groove forming an outer peripheral cutting edge 1A (G1A), the groove forming an outer peripheral cutting edge 1B (GIB), the groove forming an outer peripheral cutting edge 2A (G2A), and the grooves forming an outer peripheral cutting edge 2B (G2B)) traverse the four cutting edges (i.e. the main cutting edge 1 (112), the main cutting edge 2 (113), the sub cutting edge 1 (114), and the sub cutting edge 2 (115)) and the outer peripheral grooves (G3).

In the hollow drill bit (11) of this embodiment, the helical direction of the outer peripheral grooves (G3) is same as the twisting direction of the grooves forming outer peripheral cutting edges (i.e. the groove forming an outer peripheral cutting edge 1A (G1A), the groove forming an outer peripheral cutting edge 1B (GIB), the groove forming an outer peripheral cutting edge 2A (G2A), and the grooves forming an outer peripheral cutting edge 2B (G2B)), and the helical direction is the positive direction (in other words, the outer peripheral grooves (G3) are twisted to form a Z-shape when the tip end (TE) of the hollow drill bit (111) of this embodiment is viewed from above).

Referring to FIG. 16, the four cutting edges (i.e. the main cutting edge 1 (112), the main cutting edge 2 (113), the sub cutting edge 1 (114), and the sub cutting edge 2 (115)) have a horizontal linear shape from the outer periphery (OP) of the hollow drill bit (111) toward the center. The angle α1 of the main cutting edges (the main cutting edge 1 (112) and the main cutting edge 2 (113)) inclined toward the center, and the angle α2 of the sub cutting edge 1

(114) and the sub cutting edge 2 (115) inclined toward the center satisfy the relationship of α1=α2=0. Two cutting edges among the four cutting edges (i.e. the main cutting edge 1 (112), the main cutting edge 2 (113), the sub cutting edge 1 (114), and the sub cutting edge 2 (115)) that are formed at a distance from the center are the main cutting edges (the main cutting edge 1 (112) and the main cutting edge 2 (113)), and the angle between the main cutting edge 1 (112) and the sub cutting edge 1 (114), and the angle between the main cutting edge 2 (113) and the sub cutting edge 2 (115) may be from 90° to 100°, respectively.

Further, referring to FIG. 15, FIG. 17, and FIG. 18, the sub cutting edges (the sub cutting edge 1 (114) and the sub cutting edge 2 (115)) are provided with recesses referred to as gash pockets (GP) at the side of the rakes. These gash pockets (GP) are provided to enhance the chip-discharge ability and the drilling ability, and the rakes are formed starting from the gash pockets (GP) toward the edges of the sub cutting edge 1 (114) and the sub cutting edge 2

(115). Chips are discharged from the gash pockets (GP) to the outer peripheral grooves through the grooves forming outer peripheral cutting edges directly below the gash pockets (GP), which produces an effect of preventing chips from entering the gap (GA) (See FIG. 12 and FIG. 13 of a conventional art, in addition to FIGS. 7-1) between the hollow drill bit (111) and the small-diameter drill bit (D2) (See FIG. 11 and FIG. 12) fitted into the hollow portion of the hollow drill bit (111). This configuration produces an advantageous effect of preventing the heat generation during the drilling process and, as a result, achieving an improved cutting ability. In this embodiment, these gash pockets (GP) are provided starting from the inner periphery of the hollow drill bit (111) to the grooves forming outer peripheral cutting edges 1A (G1A) and 2A (G2A). These gash pockets (GP) do not have areas enlarging toward the inner periphery, unlike those in FIG. 13 and FIGS. 14a and 14b, but they are configured to have areas gradually enlarging from the ridge of the inner periphery toward the outer periphery. Therefore, these gash pockets (GP) can prevent chips from occupying the gap (GA).

Also, in this embodiment, the two main cutting edges mentioned above comprise the main cutting edge 1 (112) and the main cutting edge 2 (113), where the main cutting edge 1 (112) and the main cutting edge 2 (113) are formed point-symmetrically in the similar manner.

The main cutting edge 1 (112) is formed starting from the cylinder-shaped portion of the outer periphery (OP). In contrast, the main cutting edge 2 (113) is formed starting from the outer peripheral grooves (G3), and is formed to be shorter than the main cutting edge 1 (112). The main cutting edge 1 (112) serves to allow the tip end to penetrate into the bone and allow the drilling locus to form a cylinder shape during the drilling process of the hollow drill bit.

Therefore, the main cutting edge 1 prevents the hole from having a thread shape.

As exemplified in FIG. 1, the main cutting edge 1 (112) and the main cutting edge 2 (113) have linear portions with an inclined angle (Θ) inclined away from the center. Having this inclined angle (Θ), the hollow drill bit (111) in this embodiment is configured to allow chips generated during the bone-drilling process not to move in the direction toward the inner periphery of the foregoing hollow drill bit (111), but to move in the direction toward the outer periphery under the additional influence of centrifugal force when the hollow drill bit (111) in this embodiment is rotated, which generates an advantageous effect of making it difficult for chips to move to the hollow portion (H) of the hollow drill bit (111).

The two sub cutting edges mentioned above comprise the sub cutting edge 1 (114) and the sub cutting edge 2 (115), where the sub cutting edge 1 (114) and the sub cutting edge 2 (115) are positioned 90° or more rearward of the main cutting edge 1 (112) and the main cutting edge 2 (113) relative to the rotational direction of the foregoing hollow drill bit (111), respectively, and where the sub cutting edge 1 (114) and the sub cutting edge 2 (115) are formed on a same straight line in a radial direction in the cylinder-shaped portion. The cutting edges, including the main cutting edges and the sub cutting edges, may be located equi-angularly at 90° at four locations in total. Alternatively, in consideration of the creation of the gash pockets, the angles between the main cutting edges and the sub cutting edges may be set to 90°<=100°.

As shown in FIG. 20, the rake angles of the main cutting edge 1 (112) and the main cutting edge 2 (113) as well as the rake angles of the sub cutting edge 1 (114) and the sub cutting edge 2 (115) are not particularly limited. However, since a negative rake angle will cause significant damage to the cutting ability, the rake angles are set to 0° <= the rake angles of the main cutting edges < the rake angles of sub cutting edges.

The angles of the main cutting edges (112) and (113) as well as the angles of the sub cutting edges (114) and (115) of the hollow drill bit (111) in this embodiment are set to be horizontal to allow all four cutting edges to have the cutting ability. Then, the sub cutting edges 1 and 2 have rake angles set and have shapes for performing most of the drilling process.

Next, the grooves forming outer peripheral cutting edges (i.e. the grooves forming outer peripheral cutting edges 1A (G1A), the grooves forming outer peripheral cutting edges 1B (G1B), the grooves forming outer peripheral cutting edges 2A (G2A), and the grooves forming outer peripheral cutting edges 2B (G2B)) of the hollow drill bit (111) in this embodiment is described in details. As shown in FIG. 17, the grooves forming outer peripheral cutting edges comprise the grooves forming outer peripheral cutting edges 1A (G1A) and the grooves forming outer peripheral cutting edges 1B (G1B) provided at the side of the rakes of the sub cutting edges (the sub cutting edge 1 (114) and the sub cutting edge 2 (115)), and the grooves forming outer peripheral cutting edges 2A (G2A) and the grooves forming outer peripheral cutting edges 2B (G2B) provided on the side surfaces at the side of the heels. The grooves forming outer peripheral cutting edges 1A (G1A) and the grooves forming outer peripheral cutting edges 1B (G1B) are gently connected in the direction from the side of the heels of the main cutting edges (the main cutting edge 1 (112) and the main cutting edge 2 (113)) at the tip end (TE) of the hollow drill bit (111) to the distal end (DE) of the hollow drill bit (111), and are connected to the helical outer peripheral grooves (G3) continuing to the side of the rakes of the main cutting edges (the main cutting edge 1 (112) and the main cutting edge 2 (113)) with a twist toward the side of the shank, which is at the distal end (DE) of the drill bit. The grooves forming outer peripheral cutting edges 2A and 2B ((G2A) and (G2B)) have the same helix direction and the same helix angle as the grooves forming outer peripheral cutting edges 1A and 1B ((G1A) and (G2A)).

The grooves forming outer peripheral cutting edges have a depth set to be equivalent to the depth of the outer peripheral grooves (G3) or shallower than the depth of the outer peripheral grooves (G3). Large bottom surfaces are not formed. Instead, bottom surfaces with a very small diameter are formed gradually toward the rearward of the outer peripheral cutting edges. The grooves may be provided with a small volume and also with a gentle helix angle from about 5° to about 30°, for these grooves do not serve to allow chips to be discharged.

In this embodiment, most of the drilling process is performed by the sub cutting edge 1 (114) and the sub cutting edge 2 (115), and a cylinder shape is formed by the main cutting edge 1 (112), with the outer periphery (OP) of the cutting edge being corresponding to the drill diameter. The main cutting edge 2 (113) is hardly involved in the drilling process, but is present as an essential element to allow the four cutting edges to perform stable drilling during the drilling process. This produces an advantageous effect of allowing chips generated by the cutting with the sub cutting edge 1 (114), the sub cutting edge 2 (115), and the main cutting edge 1 (112) to be smoothly discharged from the outer peripheral grooves (G3) via the gash pockets (GP), the grooves forming outer peripheral cutting edges 1A (G1A), the grooves forming outer peripheral cutting edges 1B (G1B), and the grooves forming outer peripheral cutting edges 2A (G2A), for the helical outer peripheral groove (G3) are connected directly below the grooves forming outer peripheral cutting edges 2A (G2A).

When the hollow drill bit has a small diameter or a thin thickness, the chip discharging grooves (G1) like in [Embodiment 1] may not be provided. In such case, this embodiment can solve the problem.

The helical peripheral grooves (G3), the grooves forming outer peripheral cutting edges 1A (G1A), the grooves forming outer peripheral cutting edges 1B (G1B), the grooves forming outer peripheral cutting edges 2A (G2A), and the grooves forming outer peripheral cutting edges 2B (G2B), which are characteristics of the outer periphery of the hollow drill bit (111) in this embodiment, are provided to reduce the contact area between the outer periphery and the bone, for the configuration with the four cutting edges (i.e. the main cutting edge 1 (112), the main cutting edge 2 (113), the sub cutting edge 1 (114), and the sub cutting edge 2 (115)) increases the opportunity of contact between the outer peripheral cutting edges (116) and the bone. Therefore, the hollow drill bit (111) in this embodiment produces an excellent effect of reducing the friction due to the contact and suppressing the rise in temperature.

The hollow drill bit (111) in this embodiment is provided with the configuration mentioned above, and the main cutting edge 1 (112) is used to penetrate into the bone (to ensure that a hole is made with a diameter as large as the drill diameter), and then the sub cutting edges (the sub cutting edge 1 (114) and the sub cutting edge 2 (115)) are used to perform most of the drilling process, and the main cutting edge 2 (113) is used to complement the main cutting edge 1 (112) as it penetrates into the bone.

The structure from the tip end to the grooves forming outer peripheral cutting edges 1A (G1A), the grooves forming outer peripheral cutting edges 1B (G1B), the grooves forming outer peripheral cutting edges 2A (G2A), and the grooves forming outer peripheral cutting edges 2B (G2B) of the hollow drill bit (111) according to this embodiment is illustrated in FIG. 15 to FIG. 20, and has, toward the shank at the side of the distal end (DE), a pipe shape (or tube shape) with the same diameter as the drill diameter of the hollow drill bit (111), as shown in FIG. 7, or a thinner pipe shape (or tube shape) than the drill diameter, and an entire length set depending on the type of the operation. For example, FIG. 19 shows a structure on the assumption that a deep hole will be made. In a case where a deep hole will be made, the grooves forming outer peripheral cutting edges are provided throughout the long entire length, and, at the same time, the helical outer peripheral grooves are provided on a close pitch at the side of the tip end, and may be provided on an increased pitch at the side of the rearward end for this side is hardly involved in the drilling process.

### Industrial Applicability

The hollow drill bit for medical use of the present invention is to design a novel tip end shape that performs most of the drilling process so as to prevent chips from entering the gap between the hollow drill bit and the small-diameter drill bit fitted into the hollow portion of the hollow drill bit as well as to provide a structure of the outer periphery so as to allow chips to be discharged toward the outer periphery and prevent them from entering the gap portion.

### EXPLANATIONS OF REFERENCES

10 Small-diameter drill bit
11 Hollow drill bit
12 Main cutting edge 1
13 Main cutting edge 2
14 Sub cutting edge 1
15 Sub cutting edge 2
16 Outer peripheral cutting edges
111 Hollow drill bit
112 Main cutting edge 1
113 Main cutting edge 2
114 Sub cutting edge 1
115 Sub cutting edge 2
116 Outer peripheral cutting edges
AX Axial center
CD Compound drill bit
D1 Conventional hollow drill bit
D2 Conventional small-diameter drill bit
DD1 Drilling direction
DE Distal end
G1 Chip discharging grooves 1
G2 Chip discharging grooves 2
G1A Grooves forming outer peripheral cutting edges 1A
G1B Grooves forming outer peripheral cutting edges 1B
G2A Grooves forming outer peripheral cutting edges 2A
G2B Grooves forming outer peripheral cutting edges 2B
G3 Helical outer peripheral groove
GA Gap
GP Gash pocket
H Hollow portion
OP Outer periphery
RD Rotational direction
SP Starting point of drilling process
TE Tip end
Θ Inclined angle of the main cutting edge
α1 Angle inclined toward the center of the main cutting edge
α2 Angle inclined toward the center of the sub cutting edge
CS Counter sink

1. A hollow drill bit for medical use comprising
a hollow portion that penetrates an axial center in an axial direction from a tip end to a distal end; and
four cutting edges at a section of the tip end,
wherein arc-shaped outer peripheral grooves are helically formed in a cylinder-shaped outer periphery.

2. The hollow drill bit for medical use according to claim 1, wherein chip discharging grooves for discharging chips generated by the cutting edges during use of the hollow drill bit extend in a twisting manner in a direction from the tip end to the distal end of the hollow drill bit,
wherein the chip discharging grooves traverse the four cutting edges and the outer peripheral grooves, and
wherein the helical direction of the outer peripheral grooves is same as a twisting direction of the chip discharging grooves.

3. The hollow drill bit for medical use according to claim 1 , wherein the four cutting edges have a linear shape inclined from the outer periphery to a center of the hollow drill bit,
wherein two cutting edges among the four cutting edges that are formed at a distance from the center are main cutting edges, and other two cutting edges that are formed 90° or more rearward from the main cutting edges on a straight line through the center are sub cutting edges,
wherein the sub cutting edges are provided with gash pockets, and
wherein a helical direction of the outer peripheral grooves is a forward direction.

4. The hollow drill bit for medical use according to claim 1 ,wherein the two main cutting edges consist of a main cutting edge 1 and a main cutting edge 2,
wherein the main cutting edge 1 and the main cutting edge 2 are formed point-symmetrically in a same manner,
wherein the main cutting edge 1 is formed starting from a cylinder-shaped portion of the outer periphery, and
wherein the main cutting edge 2 is formed starting from the outer peripheral grooves and is formed to be shorter than the main cutting edge 1.

5. The hollow drill bit for medical use according to claim 1 ,wherein the two sub cutting edges consist of a sub cutting edge 1 and a sub cutting edge 2,
wherein the sub cutting edge 1 and the sub cutting edge 2 are positioned 90° or more rearward of the main cutting edge 1 and the main cutting edge 2 relative to a rotational direction of the hollow drill bit, and
wherein the sub cutting edge 1 and the sub cutting edge 2 are formed on a same straight line in a radial direction in the cylinder-shaped portion.

6. The hollow drill bit for medical use according to claim 1 ,wherein an angle α1 of the main cutting edges inclined toward the center and an angle α2 of the sub cutting edges inclined toward the center satisfy a relationship of 0°<α1 <α2.

7. The hollow drill bit for medical use according to claim 1,
wherein an inclined angle (Θ) made by the main cutting edge 1 and the main cutting edge 2 is formed in a direction in which chips are discharged to a side of the outer periphery due to rotation of the hollow drill bit, and as a result, chips generated by drilling process of the main cutting edge 1 are discharged from a side of a rake of the main cutting edge 1 to chip discharging grooves 1 via outer peripheral grooves,
wherein the chip discharging grooves comprise chip discharging grooves 1 provided at the side of the rakes of the sub cutting edges and chip discharging grooves 2 provided on side surfaces at a side of heels,
wherein the chip discharging grooves 1 are gently connected in the direction from the side of the heels of the main cutting edges at the tip end of the hollow drill bit to the distal end of the hollow drill bit, and are connected to the helical outer peripheral grooves continuing to the side of the rakes of the main cutting edges with a twist toward the side of the shank, which is at the distal end of the drill bit, and
wherein the chip discharging grooves 2 have a same helix direction and a same helix angle as the chip discharging grooves 1.

8. A composite drill bit comprising a small-diameter drill bit fitted into the hollow portion of the hollow drill bit according to claim 1.

9. The hollow drill bit for medical use according to claim 1, wherein grooves forming outer peripheral cutting edges with a depth equivalent to or shallower than a depth of the outer peripheral grooves extend in a twisting manner in the direction from the tip end to the distal end of the hollow drill bit,
wherein the grooves forming outer peripheral cutting edges transverse the four cutting edges and the outer peripheral grooves, and
wherein the helical direction of the outer peripheral grooves is same as the twisting direction of the grooves forming outer peripheral cutting edges.

10-13. (canceled)

14. The hollow drill bit for medical use according to claim 2, wherein the four cutting edges have a linear shape inclined from the outer periphery to a center of the hollow drill bit,
wherein two cutting edges among the four cutting edges that are formed at a distance from the center are main cutting edges, and other two cutting edges that are formed 90° or more rearward from the main cutting edges on a straight line through the center are sub cutting edges,
wherein the sub cutting edges are provided with gash pockets, and
wherein a helical direction of the outer peripheral grooves is a forward direction.

15. The hollow drill bit for medical use according to claim 2, wherein the two main cutting edges consist of a main cutting edge 1 and a main cutting edge 2, wherein the main cutting edge 1 and the main cutting edge 2 are formed point-symmetrically in a same manner,
wherein the main cutting edge 1 is formed starting from a cylinder-shaped portion of the outer periphery, and
wherein the main cutting edge 2 is formed starting from the outer peripheral grooves and is formed to be shorter than the main cutting edge 1.

16. The hollow drill bit for medical use according to claim 3, wherein the two sub cutting edges consist of a sub cutting edge 1 and a sub cutting edge 2,
wherein the sub cutting edge 1 and the sub cutting edge 2 are positioned 90° or more rearward of the main cutting edge 1 and the main cutting edge 2 relative to a rotational direction of the hollow drill bit, and
wherein the sub cutting edge 1 and the sub cutting edge 2 are formed on a same straight line in a radial direction in the cylinder-shaped portion.

17. The hollow drill bit for medical use according to claim 4, wherein the two sub cutting edges consist of a sub cutting edge 1 and a sub cutting edge 2,
wherein the sub cutting edge 1 and the sub cutting edge 2 are positioned 90° or more rearward of the main cutting edge 1 and the main cutting edge 2 relative to a rotational direction of the hollow drill bit, and
wherein the sub cutting edge 1 and the sub cutting edge 2 are formed on a same straight line in a radial direction in the cylinder-shaped portion.

18. The hollow drill bit for medical use according to claim 3, wherein an angle α1 of the main cutting edges inclined toward the center and an angle α2 of the sub cutting edges inclined toward the center satisfy a relationship of 0°<α1 <α2.

19. The hollow drill bit for medical use according to claim 4, wherein an angle α1 of the main cutting edges inclined toward the center and an angle α2 of the sub cutting edges inclined toward the center satisfy a relationship of 0°<α1 <α2.

20. The hollow drill bit for medical use according to claim 5, wherein an angle α1 of the main cutting edges inclined toward the center and an angle α2 of the sub cutting edges inclined toward the center satisfy a relationship of 0°<α1 <α2.

21. The hollow drill bit for medical use according to claim 3, wherein an inclined angle (Θ) made by the main cutting edge 1 and the main cutting edge 2 is formed in a direction in which chips are discharged to a side of the outer periphery due to rotation of the hollow drill bit, and as a result, chips generated by drilling process of the main cutting edge 1 are discharged from a side of a rake of the main cutting edge 1 to chip discharging grooves 1 via outer peripheral grooves,
wherein the chip discharging grooves comprise chip discharging grooves 1 provided at the side of the rakes of the sub cutting edges and chip discharging grooves 2 provided on side surfaces at a side of heels,
wherein the chip discharging grooves 1 are gently connected in the direction from the side of the heels of the main cutting edges at the tip end of the hollow drill bit to the distal end of the hollow drill bit, and are connected to the helical outer peripheral grooves continuing to the side of the rakes of the main cutting edges with a twist toward the side of the shank, which is at the distal end of the drill bit, and
wherein the chip discharging grooves 2 have a same helix direction and a same helix angle as the chip discharging grooves 1.

22. The hollow drill bit for medical use according to claim 4, wherein an inclined angle (Θ) made by the main cutting edge 1 and the main cutting edge 2 is formed in a direction in which chips are discharged to a side of the outer periphery due to rotation of the hollow drill bit, and as a result, chips generated by drilling process of the main cutting edge 1 are discharged from a side of a rake of the main cutting edge 1 to chip discharging grooves 1 via outer peripheral grooves,
wherein the chip discharging grooves comprise chip discharging grooves 1 provided at the side of the rakes of the sub cutting edges and chip discharging grooves 2 provided on side surfaces at a side of heels,
wherein the chip discharging grooves 1 are gently connected in the direction from the side of the heels of the main cutting edges at the tip end of the hollow drill bit to the distal end of the hollow drill bit, and are connected to the helical outer peripheral grooves continuing to the side of the rakes of the main cutting edges with a twist toward the side of the shank, which is at the distal end of the drill bit, and
wherein the chip discharging grooves 2 have a same helix direction and a same helix angle as the chip discharging grooves 1.

23. The hollow drill bit for medical use according to claim 5, wherein an inclined angle (Θ) made by the main cutting edge 1 and the main cutting edge 2 is formed in a direction in which chips are discharged to a side of the outer periphery due to rotation of the hollow drill bit, and as a result, chips generated by drilling process of the main cutting edge 1 are discharged from a side of a rake of the main cutting edge 1 to chip discharging grooves 1 via outer peripheral grooves,
wherein the chip discharging grooves comprise chip discharging grooves 1 provided at the side of the rakes of the sub cutting edges and chip discharging grooves 2 provided on side surfaces at a side of heels,
wherein the chip discharging grooves 1 are gently connected in the direction from the side of the heels of the main cutting edges at the tip end of the hollow drill bit to the distal end of the hollow drill bit, and are connected to the helical outer peripheral grooves continuing to the side of the rakes of the main cutting edges with a twist toward the side of the shank, which is at the distal end of the drill bit, and
wherein the chip discharging grooves 2 have a same helix direction and a same helix angle as the chip discharging grooves 1.

24. The hollow drill bit for medical use according to claim 6, wherein an inclined angle (Θ) made by the main cutting edge 1 and the main cutting edge 2 is formed in a direction in which chips are discharged to a side of the outer periphery due to rotation of the hollow drill bit, and as a result, chips generated by drilling process of the main cutting edge 1 are discharged from a side of a rake of the main cutting edge 1 to chip discharging grooves 1 via outer peripheral grooves,
wherein the chip discharging grooves comprise chip discharging grooves 1 provided at the side of the rakes of the sub cutting edges and chip discharging grooves 2 provided on side surfaces at a side of heels,
wherein the chip discharging grooves 1 are gently connected in the direction from the side of the heels of the main cutting edges at the tip end of the hollow drill bit to the distal end of the hollow drill bit, and are connected to the helical outer peripheral grooves continuing to the side of the rakes of the main cutting edges with a twist toward the side of the shank, which is at the distal end of the drill bit, and
wherein the chip discharging grooves 2 have a same helix direction and a same helix angle as the chip discharging grooves 1.
